(19) United States
(12) Patent Application Publication
(10) Pub. No.: US 2013/0237935 A1
(43) Pub. Date: Sep. 12, 2013

(54) Ophthalmic Drug Delivery Device with a Medicated Module

(71) Applicants: Garen Kouyoumjian, Warwickshire (GB); Malcolm Stanley Boyd, Warwickshire (GB)

(72) Inventors: Garen Kouyoumjian, Warwickshire (GB); Malcolm Stanley Boyd, Warwickshire (GB)

(73) Assignee: SanofiI-Aventis Deutschland GmbH, Frankfurt am Main (DE)

(21) Appl. No.: 13/988,588

(22) PCT Filed: Nov. 28, 2011
(86) PCT No.: PCT/EP11/71127

§ 371 Date: May 21, 2013

(30) Foreign Application Priority Data
Nov. 29, 2010 (EP) 10192980.0

(51) Int. Cl. A61F 9/00

(52) U.S. Cl. CPC A61F 9/0026
USPC 604/296; 604/294; 604/301

(57) ABSTRACT

An ophthalmic drug delivery device containing a primary medicament reservoir and a replaceable medicated module containing a second medicament where the device has a selector to allow dispense of primary medicament or a combination of the primary and secondary medicaments such that a single activation of the ophthalmic drug delivery will deliver both medicaments to the user.

### CROSS REFERENCE TO RELATED APPLICATIONS

The present application is a U.S. National Phase Application pursuant to 35 U.S.C. §371 of International Application No. PCT/EP2011/071127 filed Nov. 28, 2011, which claims priority to European Patent Application No. 10192980.0 filed Nov. 29, 2010. The entire disclosure contents of these applications are herewith incorporated by reference into the present application.

### FIELD OF INVENTION

The present application relates to medical devices and methods of delivering at least two drug agents from separate reservoirs using an ophthalmic drug delivery device having only a single activation mechanism, button or trigger and a single dispense interface. A delivery procedure initiated by the user causes a non-user settable dose (i.e., a fixed dose) of a second drug agent along with a set dose of a first drug agent to be delivered to the patient. The drug agents may be available in two or more reservoirs, containers, or packages, each containing independent (single drug compound) or pre-mixed (co-formulated multiple drug compounds) drug agents.

### BACKGROUND

Certain disease states require treatment using one or more different medicaments. Some drug compounds need to be delivered in a specific relationship with each other in order to deliver the optimum therapeutic dose. This invention is of particular benefit where combination therapy is desirable, but not possible in a single formulation for reasons such as, but not limited to, stability, compromised therapeutic performance, and/or toxicology.

A number of potential problems can arise when delivering two active medicaments or “agents” simultaneously. As just one example, the two active agents when present in a single container may interact with each other during the long-term, shelf life storage of the formulation. Therefore, there are certain advantages to storing the active components separately and then potentially combine them at the point of delivery, e.g. injection, needle-less injection, pumps, or inhalation. However, any potential process for combining the two or more agents needs to be straightforward and convenient for the user to perform reliably, repeatedly, and safely.

One further concern is that the quantities and/or proportions of each active agent making up the potential combination dose or therapy may need to be varied for each user or at different stages of their therapy. Again, as just one example, one or more active agents may require a titration period to gradually introduce a patient to a “maintenance” dose. A further example would be if one active agent requires a non-adjustable fixed dose while the other agent is varied in response to a patient's symptoms or physical condition. This potential concern could mean that pre-mixed formulations of multiple active agents may not be suitable as these pre-mixed formulations would have a fixed ratio of the active components, which could not be varied by the healthcare professional or user.

Additional concerns may arise where a multi-drug compound therapy is required, because certain users may not be able to cope with having to use more than one drug delivery system or make the necessary accurate calculation of the required dose combination. This is especially true for users with dexterity or computational difficulties.

Accordingly, there exists a strong need to provide devices and methods for the delivery of two or more medicaments in a single activation of an ophthalmic drug delivery device that is simple and safe for the user to perform and that also tends to reduce a patient's anxiety towards taking repeated doses of medicaments.

### SUMMARY

The present application discloses a medical device that enables the topical delivery to the eye of a primary ophthalmic drug with or without a fixed dose of a secondary medicament dispensed from a disposable secondary cartridge or medicated module. In particular, the device of this invention provides a means of delivering a topical dose of a secondary fluid medication from a medicated module to the cornea or sclera automatically upon delivery of a primary medicament contained within an ophthalmic drug delivery device.

The medicated module of the invention is preferably configured as a small pressurized canister that can be removably loaded into the primary device and then moved into an activation position. The medicated module of this invention provides a means by which the user is able to receive doses of two medicaments from a single activation of the primary device thus reducing their burden on storage and complexity of operation. The medicated module preferably is rotated into position such that the flow path geometry of the primary device is altered so that the flow of primary medicament goes through the medicated module and then discharges through the outlet orifice of the ophthalmic device. Such design geometry is believed to ensure good comparability between medicament delivery of the first medicament from the primary device when used in isolation, as well as when it is combined with the medicated module.

The primary ophthalmic device of this invention can dispense an ophthalmic drug in spray, liquid jet, gas or aerosol form from a primary drug cartridge (or pressurized canister). The device provides a means whereby the user can set the device to additionally dispense one or more secondary drugs contained in replaceable medicated modules in conjunction with the primary drug. This provides flexibility of therapy where more than one medicament has been prescribed for the treatment of an ocular condition.

Preferably, the primary device of the invention will accept a replaceable reservoir, preferably a pressurized canister, which contains multiple doses of a first or primary medicament. Such a pre-pressurized canister is similar in design to the canister and valve configurations in use in many pressurized metered dose inhalers (pMDI). This replaceable reservoir can be disposable or refillable. The primary drug is driven through a series of channels in the device housing to the outlet or dispense interface and onto the eye by hydraulic pressure. Pre-pressurizing the cartridge at the point of manufacture, or through mechanical or electromechanical means, such as a piston, motor or spring, with the primary device itself, could develop the needed pressure to administer the first medicament through the device. Alternatively, the primary drug could be drawn through the device using other means such as gravity or Venturi effect etc. In all cases, some form of selector mechanism that is easily manipulated by a user determines the route that the primary drug takes through the device.

In one operational state of the primary device, the drug is allowed to flow or be driven through a bypass route or flow channel in the device housing, whereby it can travel uninterrupted, except by valves or similar backflow prevention measures, to the user. Manipulation of the selector dial can alter the route taken by the primary drug to force it to pass via one or more secondary drug cavities, reservoir, or insertable medicated modules. These cavities or medicated modules may be part of the device itself or, as shown in the embodiment provided, could be disposable cartridges inserted into an aperture in the device. In a preferred embodiment these cartridges would be separately valved or otherwise sealed to maintain the integrity of the secondary medicament until driven/flushed by the travel of the primary drug out of the secondary cartridge. In addition, key junctions between the two channels would also be valved to prevent the backflow of combined drugs into the bypass channel or undiluted primary drug into the combination channel.

In one possible embodiment, the means of rerouting the primary drug is a rotating element that disengages the bypass path and engages a secondary dual drug path that allows flow to the user via a cavity intended to receive the secondary drug cartridge. Alternative methods of selection could be a linear actuation of the secondary cartridge to replace a section of the drug channel with the secondary cartridge, manipulation of internal valves or insertion of the secondary cartridge directly into the primary channel.

In one embodiment the drug delivery device comprises a housing, wherein the housing may at least partially enclose an inner housing, which may be rotatable when a selector is rotated. The inner housing may have a main flow path and a bypass flow path. The drug delivery device may be set-up to delivery only the primary medicament. The drug delivery device may be set-up to delivery the primary medicament together with the medicament from the medicated module. When the device is set-up to deliver only the primary medicament then the bypass flow path is in fluid communication with the primary medicament reservoir, e.g. a canister, and main the flow path. When the drug delivery device is set-up to deliver the primary medicament, e.g. from a canister, together with the secondary medicament from the medicated module, then the medicated module is in fluid communication with the primary medicament reservoir, e.g. a canister, and the main flow path.

The use of a replaceable medicated module makes it be possible to have a suite of different medicated modules for use in the manner described, in order to potentially treat various conditions, or to provide a range of titration options. In addition, it is possible to include more than one drug cavity in series for the dispense of several secondary drugs, or to design the secondary module itself such that a multiplicity of modules can be mounted to the primary device.

The system may also be configured to help ensure that the medicated module is only capable of being used once by a user. This may be achieved through integration of a mechanical flap on the secondary cartridge (or similar flow restriction means) that is only activated (closed off) following flushing of the secondary drug. Alternatively, the valves used to control flow of drug through the secondary drug cavity could be designed to activate only once and subsequently block fluid flow. In addition, similar mechanisms could be used to prevent insertion of a used or inappropriate cartridge into the device or to aid usability. For example, the rotation of the selector portion of the device could shield the secondary cartridge once the dual-drug path has been selected in order to prevent dislodging or removal of the cartridge while the primary drug is being dispensed.

One of the difficulties in administering ophthalmic drugs is blinking, otherwise known as the blink reflex (involuntary blinking of the eyelid(s) elicited by stimulation such as touching, air pressure or the introduction of a foreign body). The positioning of the outlet port relative to the eye (a common issue with dropper bottles and like devices) can all contribute to making drug delivery to the eye more difficult. The present invention may include features that seek to address these issues and potentially complement or improve the therapeutic performance of the system. For example, an eyepiece can be attached to the distal end of the device to assist a user in achieving a comfortable and accurate positioning of the device and the spray nozzle with respect to the target area of the cornea. Additionally, in order to overcome the natural blink reflex of the eye (typically around 0.1 s), and thereby deliver the intended dose to the surface of the eye before the lid closes, the system may be configured to deliver substantially all of the dose rapidly (e.g. in less than 75 ms from initial contact with the cornea) and in a manner that minimizes risk of damage to the corneal epithelium and sub-surface layers of the cornea (e.g. a single pulse, narrow dispersal spray, rather than a small diameter jet).

Although the invention is described and illustrated as a device designed for delivery of a liquid ophthalmic drug, the principles involved could be applied to any fluid (gas, liquid, powder) medicament for various delivery methods (injection, inhalation, oral, topical etc.).

In one embodiment of the invention there is an ophthalmic drug delivery device that comprises a housing having a single dispense interface and a main flow path in fluid communication with a primary medicament reservoir, the housing configured for attachment of a medicated module comprising a secondary medicament reservoir. The device has a selector for moving, preferably by rotation, the medicated module into the main flow path of the housing. A single activation of the ophthalmic drug delivery device causes a dose of a primary medicament and a dose of a secondary medicament to be administered to a user through the main flow path and out the dispense interface.

The drug delivery device can also include an eyecup or similar alignment device attached to the dispense interface to assist the user to accurately administer the medication. Preferably, the drug delivery device housing has a bypass flow path that is in fluid communication with only the primary medicament reservoir and the dispense interface to allow a user to administer only the primary medicament. The dispense interface can also have a spray nozzle to deliver the medicament to the eye. One more one-way valves (i.e., check valves) can be used in the main flow path to prevent back flow of medicament during dispense. The driving force for delivering the primary medicament or the combination of the primary and secondary medicaments can be supplied by the use of a pressurized canister of primary medicament configured for activation by pushing the canister in a distal direction.

In a further embodiment, the medicated module may have one or more seals on either end of reservoir containing the secondary medicament to ensure the sterility of the medicament before use.

In a preferred embodiment a master drug compound (i.e., a first medicament) is contained within a multiple dose, user-selectable device that is used with a single use, user-replaceable, module that contains a single dose of a secondary medicament and a single dispense interface or orifice. When moved into the main flow path of the housing of the drug delivery device the secondary compound is activated/delivered on dispense of the primary compound. Any number of drugs or drug combinations, such as analgesics, hormones, beta agonists or corticosteroids, or a combination of any of the above-mentioned drugs could be used with the invention.

Examples of beta agonists are, without limitation, salbutamol, levosalbutamol, terbutaline, pirbuterol, procaterol, metaproterenol, fenoterol, bitolterol mesylate, salmeterol, formoterol, bambuterol, clenbuterol, indacaterol.

Hormones are for example hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

These as well as other advantages of various aspects of the present invention will become apparent to those of ordinary skill in the art by reading the following detailed description, with appropriate reference to the accompanying drawings.

### BRIEF DESCRIPTION OF THE DRAWINGS

Exemplary embodiments are described herein with reference to the drawings, in which:

FIG. 1 illustrates a perspective view of one possible ophthalmic drug delivery device according to the present invention.

FIG. 2 illustrates possible primary medicament and secondary medicament reservoirs according to the present invention for attached to the ophthalmic device shown in FIG. 1;

FIG. 3 illustrates the ophthalmic device of FIG. 1 set to deliver only the primary medicament;

FIG. 4 illustrates the ophthalmic device of FIG. 1 set to deliver both the primary and secondary medicaments; and

FIG. 5 illustrates a sectional view of the ophthalmic device of FIG. 1 set to deliver both the primary and secondary medicaments.

### DETAILED DESCRIPTION

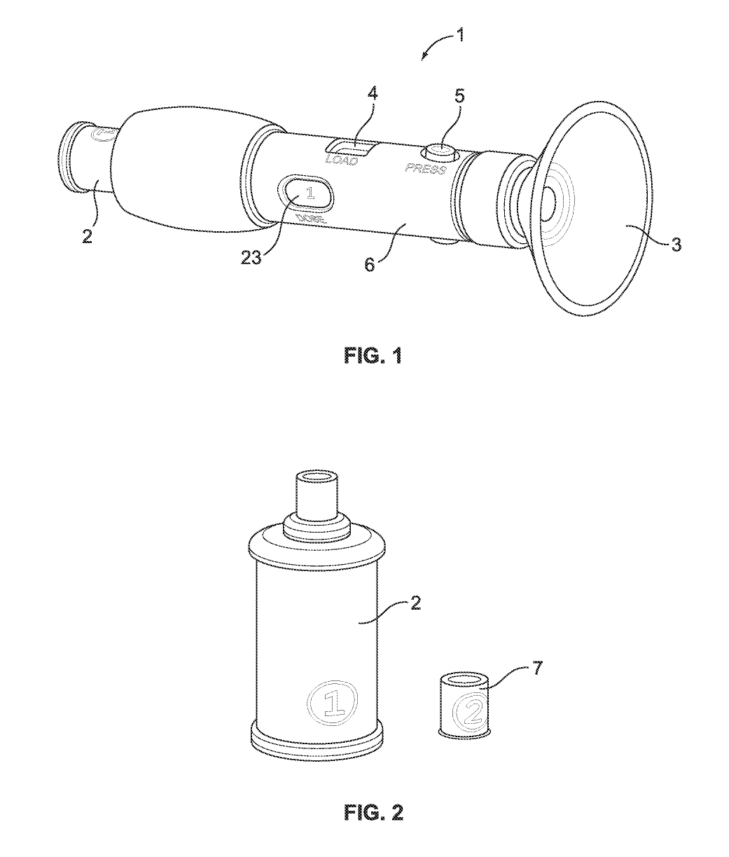

FIG. 1 illustrates one possible embodiment of the ophthalmic drug delivery device 1 of this invention. The device has a housing 6 that accepts a primary medicament reservoir, shown as a pre-pressurized canister 2 (see FIG. 2), at the proximal end of the device. At the distal end is an optional eyecup 3 to assist the user in accurately dispensing medicament to the eye. The device 1 is configured to accept a medicated module 7 (see FIG. 2) containing a secondary medicament in a secondary reservoir into loading bay 4. A single activation of canister 2, for example by pushing the canister in the distal direction along the axis of housing 6, will cause either the primary medicament contained in canister 2 alone to be dispensed or both the first and second medicaments depending on the position of selector grip 16 and as indicated by selector visual indicated in window 3. The volume and size of the dose of the second medicament is independently controlled by the design and manufacture of the reservoir 7 in the medicated module and therefore not influenced by the size of the dose generated by the activation of the primary medicament reservoir of the device. This fixed dose of the second medicament contained within the medicated module may be a single dose.

In a preferred arrangement, the drug dispense interface comprises an eyecup 3 shown in FIGS. 1 and 5, however, any orifice or discharge opening capable of passing fluids could be used. In some cases, a spray nozzle 12 (see FIG. 5) can be used in addition or separately from eyecup 3 to further assist in the deliver of medicament to the eye. Preferably, the loading bay 4 is configured such that the user can easily attach or load the module 7 to the primary device 1 and then easily remove the module after expelling the second medicament contained in the module. In certain applications, the loading bay 4 or the attachment configuration within the bay may comprise an exclusive attachment (such as a keyway) where such an exclusive attachment would only allow such a medicated module to be attached to only certain types of primary drug delivery devices and prevented from being attached to other types of ophthalmic devices or other drug delivery devices.

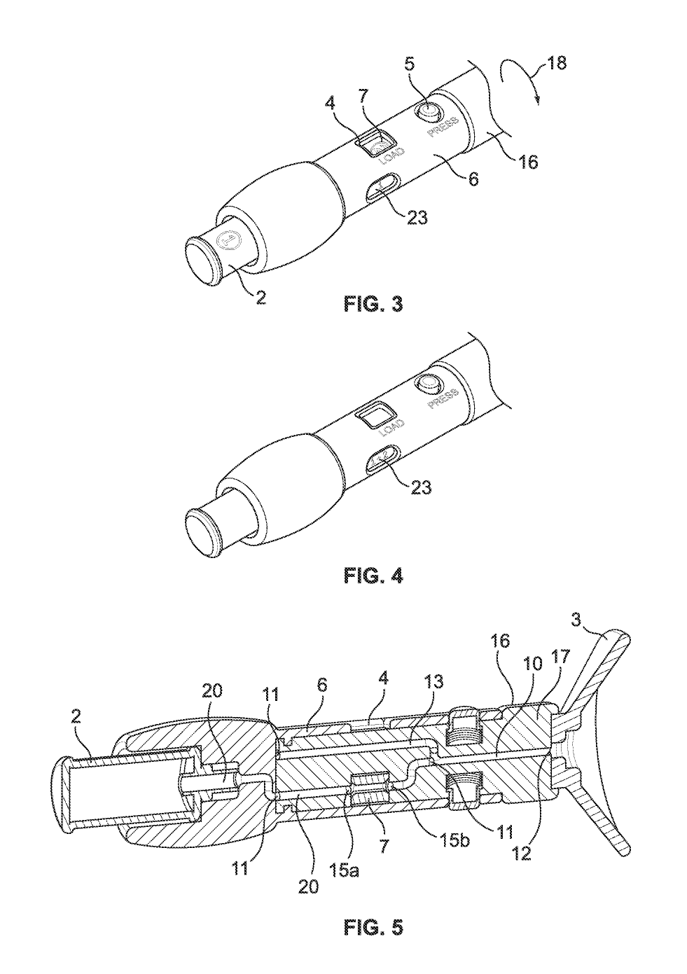

Referring now to FIG. 3, this shows device 1 immediately after module 7 has been loaded into bay 4 and the user has triggered the device selector by pushing in lock buttons 5 and rotating grip 16 in direction 18. Window 3 shows the indicator identifying that the device is initially set-up for administration of only the primary medicament contained in canister 2. FIG. 4 shows the device after completion of the selection of administration of both the primary and secondary medicaments. Window 3 now indicates that the device is ready to delivery both medicaments.

FIG. 5 shows a cross-sectional view of device 1 configured to delivery both the secondary medicament from medicated module 7 and the primary medicament from canister 2. The housing 6 partially encloses inner housing 17, which is rotatable when grip 16 is rotated in direction 18 (see FIG. 3). Inner housing 17 has a main flow path 10 and a bypass flow path 13. When the device is set-up to deliver only the primary medicament then bypass flow path 13 would be in fluid communication with discharge 20 of canister 2 and main flow path 10. When, upon rotation of the inner housing 17, the device is set-up to deliver the primary medicament from canister 2 together with the secondary medicament from the medicated module 7 then the medicated module is in fluid communication with the discharge 20 of canister 2 and the main flow path 10.

Medicated module 7 contains a reservoir configured to hold the second medicament, preferably between pressure valves 15a and 15b positioned on either side of the reservoir. The pressure valves can also act as seals to maintain sterility and prevent contamination of the medicament. The bypass and main flow paths 10 and 13 also may contain one or more one-way valves to prevent back flow during dispense.

The user operates the device 1 in exactly the same manner whether it is set-up to deliver just the primary medicament or both the primary and secondary medicaments. The user places the distal end of the device or eyecup 3 over one eye and pushes the canister 2 in a distal direction to trigger a pressurized dose of the primary medicament to flow into the main flow path, or first into the bypass flow path and then the main flow path if the device was set-up to deliver only the primary medicament. When set-up to deliver both medicaments, activation of canister 2 creates a fluid pressure in flow path 20 that then opens first pressure valve 15a of medicated module 7, pressurizing the reservoir contained therein and forcing the second medicament and primary medicament out the second valve 15b and eventually into main flow path 10. Preferably the valves 15a and 15b are configured as one-way check valves to prevent backflow in the proximal direction and possibly into container 2. In a preferred embodiment, a spray nozzle 12 is included at the outlet of main flow path 10 immediately before eyecup 3. The nozzle converts an otherwise droplet(s) of combined medicaments into a spray or aerosol. Depending on the design of the medicated module and/or the properties of the medicaments, the first medicament may be expelled with the second medicament as a mixture or may be expelled sequentially.

In one preferred arrangement, the reservoir in the medicated module comprises a single dose of the second medicament. Alternatively, the reservoir comprises a single dose of a premix of active agents or medicaments. In one preferred arrangement, the secondary medicament comprises a different type of medicament as the medicament contained within the primary medicament reservoir.

It is within the scope of the invention to configure the medicated module with a locking mechanism so as to lock and/or block the distal end, proximal end, or both after dose administration. One advantage of locking the medicated module from repeated use is that a user will be prevented from reusing an expended medicated module and therefore eliminate the possibility that a user would use the expended medicated module under the assumption that he or she is receiving the predefined dose of the primary medicament stored in a new medicated module. Likewise, such a blocking/locking feature prevents a user from re-using a non-sterile medicated module after a dose has been delivered.

The medicated module arrangements herein disclosed are preferably self-contained and may be provided as a sealed and sterile disposable module. Although not shown, the medicated modules disclosed herein could be supplied by a manufacturer contained in a protective and sterile capsule or container where the user would peel or rip open a seal or the container itself to gain access to the sterile medicated module. Alternatively, the seal could be removed automatically by the device during the action required to load or actuate the medicated module. In some instances it might be desirable to provide two or more seals for each end of the medicated module.

Moreover, in the arrangements discussed above, these arrangements have the benefit in that the second medicament is contained entirely within the medicated module, separate and away from the first medicament contained within the primary medicament reservoir. Preferably, the primary medicament reservoir is a multi-dose pre-pressurized canister similar to that used for pressurized metered dose inhalers.

Exemplary embodiments of the present invention have been described. Those skilled in the art will understand, however, that changes and modifications may be made to these embodiments without departing from the true scope and spirit of the present invention, which is defined by the claims.

1. A an ophthalmic drug delivery device comprising:
a housing having a single dispense interface and a main flow path in fluid communication with a primary medicament reservoir, the housing configured for attachment of a medicated module comprising a secondary medicament reservoir; and
a selector for moving the medicated module into the main flow path of the housing;
wherein a single activation of the ophthalmic drug delivery device causes a dose of a primary medicament and a dose of a secondary medicament to be administered to a user through the main flow path and out the dispense interface.

2. The drug delivery device of claim 1 further comprising an eyecup attached to the dispense interface.

3. The drug delivery device of claim 1 wherein the housing has a bypass flow path that is in fluid communication with only the primary medicament reservoir and the dispense interface.

4. The drug delivery device of claim 1 further comprising a spray nozzle to deliver the medicament to an eye.

5. The drug delivery device of claim 1 where the secondary medicament reservoir comprises a single dose of a secondary medicament and the primary medicament reservoir comprises multiple doses of a primary medicament.

6. The drug delivery device of claim 1 where the medicated module further comprises one-way valves located in the main flow path to prevent backflow of medicament in a proximal direction.

7. The drug delivery device of claim 1 wherein the primary medicament reservoir is a pressurized canister configured for activation by pushing the canister in a distal direction relative to the canister itself.

8. The drug delivery device of claim 1 wherein the medicated module is rotated into position such that the flow path geometry of the device is altered so that the flow of primary medicament goes through the medicated module and then discharges through the outlet orifice of the ophthalmic device.

9. The drug delivery device of claim 1, wherein the housing at least partially encloses an inner housing, which is rotatable when selector is rotated; wherein the inner housing has a main flow path and a bypass flow path.

10. The drug delivery device of claim 9, wherein when the device is set-up to deliver only the primary medicament then the bypass flow path is in fluid communication with the primary medicament reservoir and main the flow path.
